(19) United States
(12) Patent Application Publication
(10) Pub. No.: US 2023/0002761 A1
(43) Pub. Date: Jan. 5, 2023

(54) ANTI-BACTERIAL CRISPR COMPOSITIONS AND METHODS

(71) Applicant: BENSON HILL, INC., St. Louis, MO (US)

(72) Inventors: Benjamin Neil Gray, St. Louis, MO (US); Gina Christine Neumann, Manchester, MO (US)

(73) Assignee: BENSON HILL, INC., St. Louis, MO (US)

(21) Appl. No.: 17/777,833

(22) PCT Filed: Nov. 19, 2020
(86) PCT No.: PCT/IB2020/060935

§ 371 Date: May 18, 2022

Related U.S. Application Data
(60) Provisional application No. 62/937,534, filed on Nov. 19, 2019.

(51) Int. Cl. C12N 15/11; C12N 9/22; C12N 15/90; C12N 15/70

(52) U.S. Cl. CPC C12N 15/11; C12N 9/22; C12N 15/902; C12N 15/70; C12N 2310/20; C12N 2800/80

(57) ABSTRACT

Compositions and methods for targeting pre-determined DNA sequences in bacterial cells are provided. The methods result in the targeted elimination of bacterial cells that comprise the pre-determined DNA sequence(s). Compositions comprise DNA constructs comprising nucleotide sequences that encode a Cms1 protein operably linked to a promoter that is operable in the cells of interest. Methods to use these DNA constructs to selectively target and eliminate bacterial cells that harbor the targeted DNA sequence(s) are described herein.

### FIELD OF THE INVENTION

The present invention relates to compositions and methods for selectively killing bacterial cells in a sequence-specific manner.

### REFERENCE TO A SEQUENCE LISTING SUBMITTED AS A TEXT FILE VIA EFS-WEB

The official copy of the sequence listing is submitted concurrently with the specification as a text file via EFS-Web, in compliance with the American Standard Code for Information Interchange (ASCII), with a file name of BHP033P2 Sequence Listing_ST25.txt, a creation date of Oct. 15, 2020, and a size of 2,080 Kb. The sequence listing filed via EFS-Web is part of the specification and is hereby incorporated in its entirety by reference herein.

### BACKGROUND OF THE INVENTION

Various bacterial species and bacterial strains are known to cause deleterious effects in wide-ranging systems. For example, pathogenic bacteria are known to cause disease in humans, animals, and plants. These pathogenic bacteria may be found, for example, in the digestive tract, urinary tract, respiratory tract, on or in leaves, stems, root tissues, fruits, and other tissues. Furthermore, pathogenic bacteria may be transmitted by insects or other organisms. Methods to selectively eliminate these pathogenic bacteria from the organisms that are harmed by their presence are highly desirable.

Additionally, various bacterial species and bacterial strains are known to grow on crop plants and other food products. These bacteria can cause adverse effects when the food products are consumed. Methods to selectively eliminate these pathogenic bacteria from the crops and other food products that serve as hosts for these harmful bacteria are highly desirable.

Harmful bacteria are often controlled, for example, with antibiotic chemicals or with bacteriophages. These bacterial control methods are effective in many cases, but antibiotic resistant bacteria have emerged as important health threats and bacteriophage-based bacterial control methods may be hampered by an inability to sufficiently eliminate harmful bacteria. Improved methods for the elimination of harmful bacteria are highly desirable.

CRISPR systems have been proposed as a possible technology that may be adapted to selectively eliminate unwanted and/or harmful bacteria (Gomaa et al (2014) mBio e00928-13), with a focus on Type I CRISPR systems because these CRISPR systems have a processive DNase activity wherein the CRISPR nuclease hybridizes with the targeted sequence, then processively degrades DNA following this hybridization, sometimes resulting in the near complete elimination of the targeted DNA molecule (e.g., a targeted plasmid, viral DNA molecule, circular bacterial genome, or other DNA molecule). While these properties of Type I CRISPR systems may be desirable in some applications, Type I CRISPR systems also have some drawbacks. For instance, Type I CRISPR systems are typically large, multi-component systems. Their size can make packaging of Type I CRISPR systems in commonly used plasmids, viral vectors, and other vectors difficult. Furthermore, Type I CRISPR systems may not show optimal activity in some of the bacterial strains and/or species that it may be desirable to eliminate. While CRISPR systems show promise in their ability to target and eliminate undesirable bacterial strains and/or species, alternatives to Type I CRISPR systems would be valuable. Cas9-based CRISPR systems have been explored for their ability to selectively eliminate bacteria (Citorik et al (2014) Nat Biotechnol 32:1141-1145; Bikard et al (2014) Nat Biotechnol 32:1146-1150; U.S. patent application Ser. No. 14/475,785); however these systems may be hampered by the mechanism of Cas9 nucleases. Because Cas9 nucleases make a single DSB, repair of this DSB may result in survival of the bacteria.

Some Type V CRISPR enzymes have been shown to harbor a primary, sequence-specific, activity against a particular type of substrate; following this sequence-specific primary activity, the Type V enzyme is then able to access a secondary, collateral activity in a non-sequence-specific manner. As an example, Cpf1 (Cas12a) has been shown to harbor primary double-stranded break production activity against double-stranded DNA (dsDNA). After Cpf1 hybridizes with and cleaves its primary target, the protein is then capable of cleaving single-stranded DNA (ssDNA) in a non-sequence-specific manner (Chen et al (2018) Science 360:436-439). Other Type V CRISPR enzymes have been shown, for example, to harbor a primary activity against RNA, with secondary activities directed against RNA and ssDNA (Yan et al (2019) Science 363:88-91). The present invention describes the primary and secondary activities of Cms1 enzymes, a group of Type V CRISPR enzymes whose secondary activities can result in bacterial cell death.

### SUMMARY OF THE INVENTION

Compositions and methods for selectively killing bacterial cells using Cms1 CRISPR systems are provided. The methods result in cell death for those bacterial cells that harbor particular pre-determined and targeted DNA sequences leaving other bacterial cells that do not comprise the targeted DNA sequences unharmed. Compositions comprise DNA constructs comprising nucleotide sequences that encode a Cms1 protein operably linked to a promoter that is operable in the cells of interest. In some embodiments, the compositions further comprise nucleotide sequences that encode at least one guide RNA that can interact with a Cms1 protein of the invention and can guide the Cms1 protein to bind with a pre-determined DNA sequence. In some embodiments, the compositions are part of a bacteriophage that is capable of infecting the bacterial cell(s) of interest. The DNA constructs comprising polynucleotide sequences that encode the Cms1 proteins of the invention, or the Cms1 proteins of the invention themselves, can be used to direct the Cms1 protein to hybridize with genomic DNA in a bacterial cell or cells of interest at pre-determined genomic loci, with this hybridization in turn leading to Cms1-mediated cell death. Methods to use these DNA constructs to selectively target and eliminate bacterial strains and/or species are described herein.

### BRIEF DESCRIPTION OF THE DRAWINGS

FIG. 1 shows a phylogenetic tree drawn from a MUSCLE alignment of the Cms1 protein sequences of the present invention. Sm-type, Sulf-type, and Unk40-type Cms1 nucleases are indicated by black, red, and blue lines, respectively.

FIG. 2 shows a summary of amino acid motifs shared among Sm-type Cms1 proteins. The weblogo figures in boxes 1-10 correspond to SEQ ID NOs:1-10, respectively, and their locations on the SmCms1 protein (SEQ ID NO:41) are shown.

FIG. 3 shows a summary of amino acid motifs shared among Sulf-type Cms1 proteins. The weblogo figures in boxes 1-17 correspond to SEQ ID NOs: 11-27, respectively, and their locations on the SulfCms1 protein (SEQ ID NO:42) are shown.

FIG. 4 shows a summary of amino acid motifs shared among Unk40-type Cms1 proteins. The weblogo figures in boxes 1-7 correspond to SEQ ID NOs: 28-34, respectively, and their locations on the Unk40Cms1 protein (SEQ ID NO:88) are shown.

### DETAILED DESCRIPTION OF THE INVENTION

Methods and compositions are provided herein for the selective targeting and elimination of bacterial species and/or bacterial strains that harbor certain pre-determined DNA sequences through the use of the CRISPR-Cms system and components thereof. The CRISPR enzymes of the invention are selected from a Cms enzyme, e.g. a Cms1 ortholog or a mutated Cms1 enzyme. Cms1 is an abbreviation for CRISPR from Microgenomates and Smithella, and is so named because some bacterial species in these groups encode Cms1 nucleases; the terms Csm1 and Cms1 may be used interchangeably. Cms1 nucleases may also be referred to as Cas12f nucleases. The methods and compositions include nucleic acids to bind target DNA sequences. This is advantageous as nucleic acids are much easier and less expensive to produce than, for example, peptides, and the specificity can be varied according to the length of the stretch where homology is sought. Complex 3-D positioning of multiple fingers, for example is not required. In some preferred embodiments, the nucleic acids are guide polynucleotides such as guide RNAs (gRNAs; alternatively CRISPR RNAs or crRNAs) that are capable of interacting with a Cms1 enzyme and of hybridizing with a DNA sequence through base pairing. As used herein, guide RNAs that are capable of interacting or that are designed to interact with a Cms1 polypeptide can bind, associate with, or otherwise form a complex with the Cms1 polypeptide. Methods of measuring interaction of gRNAs with Cms1 polypeptide are well known in the art.

Also provided are nucleic acids encoding the Cms1 polypeptides, as well as methods of using Cms1 polypeptides to target specific DNA sequences of bacterial host cells. The targeted DNA sequences may be present in genomic DNA, plasmid DNA, or other DNA elements harbored within the targeted bacterial cells. The Cms1 polypeptides interact with specific guide polynucleotides such as guide RNAs (gRNAs), which direct the Cms1 endonuclease to a specific target site. Without being limited by theory, the Cms1-gRNA complex hybridizes with the targeted DNA sequence (the “initial hybridization event”), at which site the Cms1 endonuclease introduces a double-stranded break (DSB). This process of hybridization and DSB production leads to a change in the structure of the Cms1 protein, resulting in a protein that is capable of degrading double-stranded DNA (dsDNA) and/or RNA in a non-sequence-specific manner, leading to cell death. Since the specificity of the initial hybridization event is provided by the guide RNA, the Cms1 polypeptide is universal and can be used with different guide RNAs to target different genomic sequences. Cms1-associated CRISPR arrays are processed into mature crRNAs without the requirement of an additional trans-activating crRNA (tracrRNA). Cms1 proteins can process crRNA arrays that include multiple spacer sequences; the compositions of the invention include, in some embodiments, crRNA arrays with multiple spacer sequences designed to target multiple different loci within the bacterial strain and/or bacterial species of interest. Cms1-gRNA systems can target DNA sequences adjacent to a variety of protospacer-adjacent motif (PAM) sequences, with the PAM sequence located immediately 5′ of the DNA sequence targeted by Cms1. The initial hybridization event is sequence-specific with limited off target effects, resulting in sequence-specific killing of bacterial cells of interest without harming bacterial cells that do not harbor the sequence(s) of interest.

### I. Cms1 Endonucleases and Guide Polynucleotides

Provided herein are Cms1 endonucleases, and fragments and variants thereof, for use in targeting sequences within bacterial cells, for example within genomic DNA, plasmids, or other DNA-containing elements found in bacterial cells. As used herein, the term Cms1 endonucleases or Cms1 polypeptides refers to homologs, orthologs, and variants of the Cms1 polypeptide sequences set forth in SEQ ID NOs:41-160 and 340-341. Typically, Cms1 endonucleases can act without the use of tracrRNAs, requiring on a single gRNA for sequence specificity. In general, a Cms1-gRNA complex can perform an initial hybridization event to target a particular sequence. Without being limited by theory, following this initial hybridization event, the Cms1 protein is then able to perform a secondary collateral activity directed against double-stranded DNA (dsDNA) or RNA without any sequence specificity. This collateral activity results in cell death in those cells in which the Cms1-gRNA complex undergoes an initial hybridization event. In general, Cms1 polypeptides comprise at least one RNA recognition and/or RNA binding domain. RNA recognition and/or RNA binding domains interact with guide RNAs. Typically the guide RNA comprises a region with a stem-loop structure that interacts with the Cms1 polypeptide. This stem-loop often comprises the sequence UCUACN3-5GUAGAU (SEQ ID NOs:35-37, encoded by SEQ ID NOs:38-40), with “UCUAC” and “GUAGA” base-pairing to form the stem of the stem-loop. N3-5 denotes that any base may be present at this location, and 3, 4, or 5 nucleotides may be included at this location. Some CRISPR nucleases have been shown to function with guide polynucleotides in which some of the ribonucleotide residues have been replaced by deoxyribonucleotide residues (Yin et al (2018) Nat Chem Biol 14:311-316; U.S. Pat. No. 9,650,617); the present invention also encompasses embodiments in which the guide polynucleotide is a guide RNA, embodiments in which the guide polynucleotide is a guide DNA, and embodiments in which the guide polynucleotide comprises both DNA and RNA residues. In specific embodiments, a Cms1 polypeptide, or a polynucleotide encoding a Cms1 polypeptide, comprises: an RNA-binding portion that interacts with the DNA-targeting RNA, and an activity portion that exhibits site-directed enzymatic activity, such as a RuvC endonuclease domain. Without being limited by theory, the RuvC endonuclease domain may also exhibit secondary, collateral activity directed against dsDNA and/or RNA in a non-sequence-specific manner.

Cms1 polypeptides can be wild type Cms1 polypeptides, modified Cms1 polypeptides, or a fragment of a wild type or modified Cms1 polypeptide. The Cms1 polypeptide can be modified to increase nucleic acid binding affinity and/or specificity, alter an enzymatic activity, and/or change another property of the protein. For example, nuclease (i.e., DNase, RNase) domains of the Cms1 polypeptide can be modified, deleted, or inactivated. Alternatively, the Cms1 polypeptide can be modified or truncated to alter or remove domains that are not essential for the function of the protein.

In some embodiments, the Cms1 polypeptide can be derived from a wild type Cms1 polypeptide or fragment thereof. In other embodiments, the Cms1 polypeptide can be derived from a modified Cms1 polypeptide. For example, the amino acid sequence of the Cms1 polypeptide can be modified to alter one or more properties (e.g., nuclease activity, affinity, stability, solubility, etc.) of the protein.

In general, a Cms1 polypeptide comprises at least one nuclease domain, but need not contain an HNH domain such as the one found in Cas9 proteins. For example, a Cms1 polypeptide can comprise a RuvC or RuvC-like nuclease domain. Without being limited by theory, the RuvC or RuvC-like domain may comprise three catalytic residues that are typically aspartate, glutamate, and aspartate, respectively, and may be responsible for the Cms1 nuclease activity.

In some embodiments, the Cms1 polypeptide can comprise at least one cell-penetrating domain. The cell-penetrating domain can be located at the N-terminus, the C-terminus, or in an internal location of the protein.

In still other embodiments, the Cms1 polypeptide can also comprise at least one marker domain. Non-limiting examples of marker domains include fluorescent proteins, purification tags, and epitope tags. In certain embodiments, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellowl), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyanl, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In other embodiments, the marker domain can be a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, 51, T7, V5, VSV-G, 6×His, biotin carboxyl carrier protein (BCCP), and calmodulin.

In certain embodiments, the Cms1 polypeptide may be part of a protein-RNA complex comprising a guide polynucleotide. In some embodiments, the guide polynucleotide may be a guide RNA. The guide polynucleotide interacts with the Cms1 polypeptide to direct the Cms1 polypeptide to a specific target site in a bacterial cell, where the target site comprises dsDNA that may be present in genomic DNA, plasmid DNA, or other DNA components in a bacterial cell of interest. If a suitable protospacer-adjacent motif (PAM) sequence is present immediately 5′ of the target sequence, the Cms1-guide polynucleotide complex may hybridize with the dsDNA target sequence. Following this initial hybridization event, the Cms1 enzyme may cleave the target DNA. Without being limited by theory, the Cms1 enzyme may then undergo a structural change that may allow the Cms1 enzyme to cleave dsDNA and/or RNA in a non-sequence-specific manner (“secondary” or “collateral” activity). This secondary activity may result in bacterial cell death. As used herein, the term “DNA-targeting RNA” refers to a guide RNA that interacts with the Cms1 polypeptide and the target site of the nucleotide sequence of interest in the genome of a plant cell. A DNA-targeting RNA, or a DNA polynucleotide encoding a DNA-targeting RNA, can comprise: a first segment comprising a nucleotide sequence that is complementary to a sequence in the target DNA, and a second segment that interacts with a Cms1 polypeptide.

Cms proteins for use in the invention include, but are not limited to, Cms1 proteins that comprise at least one amino acid motif selected from the group consisting of SEQ ID NOs:1-10. In other embodiments, a Cms1 protein comprises at least one amino acid motif selected from the group consisting of SEQ ID NOs:11-27. In other embodiments, a Cms1 protein comprises at least one amino acid motif selected from the group consisting of SEQ ID NOs:28-34. In certain preferred embodiments, a Cms1 protein comprises more than one amino acid motif selected from the group consisting of SEQ ID NOs:1-10. In certain preferred embodiments, a Cms1 protein comprises more than one amino acid motif selected from the group consisting of SEQ ID NOs:11-27. In certain preferred embodiments, a Cms1 protein comprises more than one amino acid motif selected from the group consisting of SEQ ID NOs:28-34. Particular Cms1 protein sequences are set forth in SEQ ID NOs:41-160 and 340-341; particular Cms1 protein-encoding polynucleotide sequences are set forth in SEQ ID NOs:161-317 and 342-343. In certain preferred embodiments, a Cms1 protein has at least about 80% identity with a sequence selected from the group consisting of SEQ ID NOs:41-160 and 340-341.

The polynucleotides encoding Cms1 polypeptides disclosed herein can be used to isolate corresponding sequences from other prokaryotic or eukaryotic organisms, or from metagenomically-derived sequences whose native host organism is unclear or unknown. In this manner, methods such as PCR, hybridization, and the like can be used to identify such sequences based on their sequence homology or identity to the sequences set forth herein. Sequences isolated based on their sequence identity to the entire Cms1 sequences set forth herein or to variants and fragments thereof are encompassed by the present invention. Such sequences include sequences that are orthologs of the disclosed Cms1 sequences. “Orthologs” is intended to mean genes derived from a common ancestral gene and which are found in different species as a result of speciation. Genes found in different species are considered orthologs when their nucleotide sequences and/or their encoded protein sequences share at least about 75%, about 80%, about 85%, about 90%, about 91%, about 92%, about 93%, about 94%, about 95%, about 96%, about 97%, about 98%, about 99%, or greater sequence identity. Functions of orthologs are often highly conserved among species. Thus, isolated polynucleotides that encode polypeptides having Cms1 endonuclease activity and which share at least about 75% or more sequence identity to the sequences disclosed herein, are encompassed by the present invention.

Fragments and variants of the Cms1 polynucleotides and Cms1 amino acid sequences encoded thereby that retain Cms1 nuclease activity are encompassed herein. By “Cms1 nuclease activity” is intended the binding of and hybridization with a pre-determined DNA sequence (the “targeted sequence”) as mediated by a guide RNA. Cms1 nuclease activity can further comprise double-strand break production of the targeted sequence (“primary activity”), and can further comprise non-sequence-specific nuclease activity directed against dsDNA and/or RNA (“secondary activity”) following the primary activity. By “fragment” is intended a portion of the polynucleotide or a portion of the amino acid sequence. “Variants” is intended to mean substantially similar sequences. For polynucleotides, a variant comprises a polynucleotide having deletions (i.e., truncations) at the 5′ and/or 3′ end; deletion and/or addition of one or more nucleotides at one or more internal sites in the native polynucleotide; and/or substitution of one or more nucleotides at one or more sites in the native polynucleotide. As used herein, a “native” polynucleotide or polypeptide comprises a naturally occurring nucleotide sequence or amino acid sequence, respectively. Generally, variants of a particular polynucleotide of the invention will have at least about 75%, 80%, 85%, 90%, 91%, 92%, 93%, 94%, 95%, 96%, 97%, 98%, 99% or more sequence identity to that particular polynucleotide as determined by sequence alignment programs and parameters as described elsewhere herein.

“Variant” amino acid or protein is intended to mean an amino acid or protein derived from the native amino acid or protein by deletion (so-called truncation) of one or more amino acids at the N-terminal and/or C-terminal end of the native protein; deletion and/or addition of one or more amino acids at one or more internal sites in the native protein; or substitution of one or more amino acids at one or more sites in the native protein. Variant proteins encompassed by the present invention are biologically active, that is they continue to possess the desired biological activity of the native protein. Biologically active variants of a native polypeptide will have at least about 80%, 85%, 90%, 91%, 92%, 93%, 94%, 95%, 96%, 97%, 98%, 99% or more sequence identity to the amino acid sequence for the native sequence as determined by sequence alignment programs and parameters described herein. A biologically active variant of a protein of the invention may differ from that protein by as few as 1-15 amino acid residues, as few as 1-10, such as 6-10, as few as 5, as few as 4, 3, 2, or even 1 amino acid residue.

Variant sequences may also be identified by analysis of existing databases of sequenced genomes. In this manner, corresponding sequences can be identified and used in the methods of the invention.

Methods of alignment of sequences for comparison are well known in the art. Thus, the determination of percent sequence identity between any two sequences can be accomplished using a mathematical algorithm. Non-limiting examples of such mathematical algorithms are the algorithm of Myers and Miller (1988) CABIOS 4:11-17; the local alignment algorithm of Smith et al. (1981) Adv. Appl. Math. 2:482; the global alignment algorithm of Needleman and Wunsch (1970) J. Mol. Biol. 48:443-453; the search-for-local alignment method of Pearson and Lipman (1988) Proc. Natl. Acad. Sci. 85:2444-2448; the algorithm of Karlin and Altschul (1990) Proc. Natl. Acad. Sci. USA 87:2264-2268, modified as in Karlin and Altschul (1993) Proc. Natl. Acad. Sci. USA 90:5873-5877.

Computer implementations of these mathematical algorithms can be utilized for comparison of sequences to determine sequence identity. Such implementations include, but are not limited to: CLUSTAL in the PC/Gene program (available from Intelligenetics, Mountain View, Calif.); the ALIGN program (Version 2.0) and GAP, BESTFIT, BLAST, FASTA, and TFASTA in the GCG Wisconsin Genetics Software Package, Version 10 (available from Accelrys Inc., 9685 Scranton Road, San Diego, Calif., USA). Alignments using these programs can be performed using the default parameters. The CLUSTAL program is well described by Higgins et al. (1988) Gene 73:237-244; Higgins et al. (1989) CABIOS 5:151-153; Corpet et al. (1988) Nucleic Acids Res. 16:10881-90; Huang et al. (1992) CABIOS 8:155-65; and Pearson et al. (1994) Meth. Mol. Biol. 24:307-331. The ALIGN program is based on the algorithm of Myers and Miller (1988) supra. A PAM120 weight residue table, a gap length penalty of 12, and a gap penalty of 4 can be used with the ALIGN program when comparing amino acid sequences. The MUSCLE algorithm for multiple sequence alignment may be used for comparisons of multiple nucleic acid or protein sequences (Edgar (2004) Nucleic Acids Research 32:1792-1797). The BLAST programs of Altschul et al (1990) J. Mol. Biol. 215:403 are based on the algorithm of Karlin and Altschul (1990) supra. BLAST nucleotide searches can be performed with the BLASTN program, score=100, wordlength=12, to obtain nucleotide sequences homologous to a nucleotide sequence encoding a protein of the invention. BLAST protein searches can be performed with the BLASTX program, score=50, wordlength=3, to obtain amino acid sequences homologous to a protein or polypeptide of the invention. To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al. (1997) supra. When utilizing BLAST, Gapped BLAST, PSI-BLAST, the default parameters of the respective programs (e.g., BLASTN for nucleotide sequences, BLASTX for proteins) can be used. See the website at www.ncbi.nlm.nih.gov. Alignment may also be performed manually by inspection.

The nucleic acid molecules encoding Cms1 polypeptides, or fragments or variants thereof, can be codon optimized for expression in a bacterial cell or organism of interest. A “codon-optimized gene” is a gene having its frequency of codon usage designed to mimic the frequency of preferred codon usage of the host cell. Nucleic acid molecules can be codon optimized, either wholly or in part. Because any one amino acid (except for methionine and tryptophan) is encoded by a number of codons, the sequence of the nucleic acid molecule may be changed without changing the encoded amino acid. Codon optimization is when one or more codons are altered at the nucleic acid level such that the amino acids are not changed but expression in a particular host organism is increased. Those having ordinary skill in the art will recognize that codon tables and other references providing preference information for a wide range of organisms are available in the art (see, e.g., Zhang et al. (1991) Gene 105:61-72; Murray et al. (1989) Nucl. Acids Res. 17:477-508).

In some embodiments, DNA encoding the Cms1 polypeptides of the invention, and DNA encoding guide polynucleotide(s) of the invention, may be included as part of a bacteriophage or modified bacteriophage, or may be included as part of a plasmid (for example a conjugative plasmid), phagemid, cosmid, or other DNA molecule capable of replication in a bacterial cell or cells of interest. The terms phage and bacteriophage may be used interchangeably. In some embodiments, a phage or a phagemid derived from M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage or 186 phage may be used to deliver a polynucleotide encoding a Cms1 polypeptide of the invention and/or one or more guide polynucleotide(s) of the invention, to the bacterial cell(s) of interest. Bacteriophage may be engineered, for example, to have a broad or narrow host range using methods known in the art (Yehl et al 2019 BioRxiv dx.doi.org/10.1101/699090).

### II. Nucleic Acids Encoding Cms1 Polypeptides

Nucleic acids encoding any of the Cms1 polypeptides or fusion proteins described herein are provided. The nucleic acid can be RNA or DNA. Examples of polynucleotides that encode Cms1 polypeptides are set forth in the group consisting of SEQ ID NOs:161-317 and 342-343. In one embodiment, the nucleic acid encoding the Cms1 polypeptide is mRNA. The mRNA can be 5′ capped and/or 3′ polyadenylated. In another embodiment, the nucleic acid encoding the Cms1 polypeptide is DNA. The DNA can be present in a phage, plasmid, or other vector.

Nucleic acids encoding the Cms1 polypeptide or fusion proteins can be codon optimized for efficient translation into protein in the cell of interest. Programs for codon optimization are available in the art (e.g., OPTIMIZER at genomes.urv.es/OPTIMIZER; OptimumGene™ from GenScript at www.genscript.com/codon_opt.html).

In certain embodiments, DNA encoding the Cms1 polypeptide can be operably linked to at least one promoter sequence. The DNA coding sequence can be operably linked to a promoter control sequence for expression in a host cell of interest, for example a bacterial cell. “Operably linked” is intended to mean a functional linkage between two or more elements. For example, an operable linkage between a promoter and a coding region of interest (e.g., region coding for a Cms1 polypeptide or guide RNA) is a functional link that allows for expression of the coding region of interest. Operably linked elements may be contiguous or non-contiguous. When used to refer to the joining of two protein coding regions, by operably linked is intended that the coding regions are in the same reading frame.

The promoter sequence can be derived from bacterial sequences, viral sequences, synthetically-designed sequences, or other sources. It is recognized that different applications can be enhanced by the use of different promoters in the nucleic acid molecules to modulate the timing, location and/or level of expression of the Cms1 polypeptide and/or guide RNA. Such nucleic acid molecules may also contain, if desired, a promoter regulatory region (e.g., one conferring inducible, constitutive, or environmentally- or developmentally-regulated expression), a transcription initiation start site, a ribosome binding site, an RNA processing signal, a transcription termination site, and/or a polyadenylation signal.

The nucleic acid sequences encoding the Cms1 polypeptide can be operably linked to a promoter sequence that is recognized by a phage RNA polymerase for in vitro mRNA synthesis. In such embodiments, the in vitro-transcribed RNA can be purified for use in the methods of genome modification and/or bacterial cell elimination described herein. For example, the promoter sequence can be a T7, T3, or SP6 promoter sequence or a variation of a T7, T3, or SP6 promoter sequence. In some embodiments, the sequence encoding the Cms1 polypeptide can be operably linked to a promoter sequence for in vitro expression of the Cms1 polypeptide. In such embodiments, the expressed protein and/or guide polynucleotide such as a guide RNA can be purified for use in the methods described herein.

The DNA encoding the Cms1 polypeptide or fusion protein can be present in a vector. Suitable vectors include engineered bacteriophages, plasmid vectors (for example conjugative plasmid vectors), phagemids, cosmids, artificial/mini-chromosomes, transposons, and viral vectors (e.g., lentiviral vectors, adeno-associated viral vectors, etc.). In one embodiment, the DNA encoding the Cms1 polypeptide is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001. In some embodiments, the DNA encoding the Cms1 polypeptide is present in an engineered bacteriophage, where the native bacteriophage sequence is derived from a bacteriophage that is capable of infecting the bacterial cell(s) of interest.

In some embodiments, the expression vector comprising the sequence encoding the Cms1 polypeptide can further comprise a sequence encoding a guide RNA. The sequence encoding the guide RNA can be operably linked to at least one transcriptional control sequence for expression of the guide RNA in the cell of interest.

### III. Methods for Targeting a Nucleotide Sequence in a Bacterial Cell or Cells

Methods are provided herein for targeting a nucleotide sequence in a bacterial cell. The methods comprise introducing into a bacterial cell one or more DNA-targeting polynucleotides such as, for example, a DNA-targeting RNA (“guide RNA,” “gRNA,” “CRISPR RNA,” or “crRNA”) or a DNA polynucleotide encoding a DNA-targeting RNA, wherein the DNA-targeting polynucleotide comprises: (a) a first segment comprising a nucleotide sequence that is complementary to a sequence in the target DNA; and (b) a second segment that interacts with a Cms1 polypeptide and also introducing to the bacterial cell a Cms1 polypeptide, or a polynucleotide such as a DNA molecule or an RNA molecule encoding a Cms1 polypeptide, wherein the a Cms1 polypeptide comprises: (a) a polynucleotide-binding portion that interacts with the gRNA or other DNA-targeting polynucleotide; and (b) an activity portion that may comprise a catalytic domain such as a RuvC domain that exhibits site-directed enzymatic activity. In some embodiments, these methods result in the partial or complete killing and elimination of the bacterial cell or cells into which the Cms1 or encoding polynucleotide and guide polynucleotide have been introduced. For example, the methods described herein can result in a 5%, 10%, 15%, 20%, 25%, 30%, 50%, 75%, 80%, 85%, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more decrease in the viable bacterial population in which the Cms1 or encoding polynucleotide and guide polynucleotide have been introduced. Bacterial cell viability can be measured by any method known in the art, including plate count (e.g., CFU, CFU/g, CFU/mL), turbidity measurement, cell lysis, or any other method known in the art. In specific embodiments, bacterial cell killing as used herein refers to a bacteriostatic elimination of future bacterial growth.

The methods disclosed herein comprise introducing into a bacterial cell at least one Cms1 polypeptide or a nucleic acid encoding at least one Cms1 polypeptide, as described herein. In some embodiments, the Cms1 polypeptide can be introduced into the bacterial cell as an isolated protein. In such embodiments, the Cms1 polypeptide can further comprise at least one cell-penetrating domain, which facilitates cellular uptake of the protein. In some embodiments, the Cms1 polypeptide can be introduced into the bacterial cell as a nucleoprotein in complex with a guide polynucleotide (for instance, as a ribonucleoprotein in complex with a guide RNA). In other embodiments, the Cms1 polypeptide can be introduced into the genome host as an mRNA molecule that encodes the Cms1 polypeptide. In still other embodiments, the Cms1 polypeptide can be introduced into the bacterial cell or cells as a DNA molecule comprising an open reading frame that encodes the Cms1 polypeptide. In general, DNA sequences encoding the Cms1 polypeptide or fusion protein described herein are operably linked to a promoter sequence that will function in the bacterial cell or cells of interest. The DNA sequence can be linear, or the DNA sequence can be part of a vector. In still other embodiments, the Cms1 polypeptide can be introduced into the bacterial cell or cells as an RNA-protein complex comprising the guide RNA. In certain embodiments, the Cms1 polypeptide, Cms1-gRNA ribonucleoprotein complex, and/or Cms1-encoding polynucleotide can be introduced into the bacterial cell or cells of interest via nanoparticle-aided transformation (Kumari et al 2017 FEMS Microbiol Lett 364:fnx081; French 2019 BioRxiv dx.doi.org/10.1101/559252).

In certain embodiments, DNA encoding the Cms1 polypeptide can further comprise a sequence encoding one or more guide RNAs. In general, each of the sequences encoding the Cms1 polypeptide and the guide RNA(s) is operably linked to one or more appropriate promoter sequences that enable expression of the Cms1 polypeptide and the guide RNA(s), respectively, in the bacterial cell or cells of interest. The DNA sequence encoding the Cms1 polypeptide and the guide RNA(s) can further comprise additional expression control, regulatory, and/or processing sequence(s). The DNA sequence encoding the Cms1 polypeptide and the guide RNA(s) can be linear or can be part of a vector.

Methods described herein further can also comprise introducing into a bacterial cell or cells at least one guide RNA or DNA encoding at least one polynucleotide such as a guide RNA. A guide RNA interacts with the Cms1 polypeptide to direct the Cms1 polypeptide to a specific target site, at which site the guide RNA base pairs with a specific DNA sequence in the targeted site. Guide RNAs can comprise three regions: a first region that is complementary to the target site in the targeted DNA sequence, a second region that forms a stem loop structure, and a third region that remains essentially single-stranded. The first region of each guide RNA is different such that each guide RNA guides a Cms1 polypeptide to a specific target site. The second and third regions of each guide RNA can be the same in all guide RNAs.

One region of the guide RNA is complementary to a sequence (i.e., protospacer sequence) at the target site in the targeted DNA such that the first region of the guide RNA can base pair with the target site. In various embodiments, the first region of the guide RNA can comprise from about 8 nucleotides to more than about 30 nucleotides. For example, the region of base pairing between the first region of the guide RNA and the target site in the nucleotide sequence can be about 8, about 9, about 10, about 11, about 12, about 13, about 14, about 15, about 16, about 17, about 18, about 19, about 20, about 22, about 23, about 24, about 25, about 27, about 30 or more than 30 nucleotides in length. In an exemplary embodiment, the first region of the guide RNA is about 20, 21, 22, 23, 24, or 25 nucleotides in length. The guide RNA also can comprise a second region that forms a secondary structure. In some embodiments, the secondary structure comprises a stem or hairpin. The length of the stem can vary. For example, the stem can range from about 5, to about 6, to about 10, to about 15, to about 20, to about 25 base pairs in length. The stem can comprise one or more bulges of 1 to about 10 nucleotides. In some preferred embodiments, the hairpin structure comprises the sequence UCUACN3-5GUAGAU (SEQ ID NOs:35-37, encoded by SEQ ID NOs:38-40), with “UCUAC” and “GUAGA” base-pairing to form the stem. “N3-5” indicates 3, 4, or 5 nucleotides. Thus, the overall length of the second region can range from about 14 to about 25 nucleotides in length. In certain embodiments, the loop is about 3, 4, or 5 nucleotides in length and the stem comprises about 5, 6, 7, 8, 9, or 10 base pairs.

The guide RNA can also comprise a third region that remains essentially single-stranded. Thus, the third region has no complementarity to any nucleotide sequence in the cell of interest and has no complementarity to the rest of the guide RNA. The length of the third region can vary. In general, the third region is more than about 4 nucleotides in length. For example, the length of the third region can range from about 5 to about 60 nucleotides in length. The combined length of the second and third regions (also called the universal or scaffold region) of the guide RNA can range from about 30 to about 120 nucleotides in length. In one aspect, the combined length of the second and third regions of the guide RNA range from about 40 to about 45 nucleotides in length.

In a preferred embodiment, the guide RNA comprises a single molecule comprising all three regions. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 25 nucleotides) that base pairs to the other sequence to form a functional guide RNA. In specific embodiments, the guide RNA is a single molecule (i.e., crRNA) that interacts with the target site in the chromosome and the Cms1 polypeptide without the need for a second guide RNA (i.e., a tracrRNA).

In certain embodiments, the guide RNA(s) can be introduced into the bacterial cell as an RNA molecule. The RNA molecule can be transcribed in vitro. Alternatively, the RNA molecule can be chemically synthesized. In other embodiments, the guide RNA can be introduced into the genome host as a DNA molecule that encodes the guide RNA. In such cases, the DNA encoding the guide RNA can be operably linked to one or more promoter sequences for expression of the guide RNA in the bacterial cell or cells of interest.

In some embodiments, multiple guide RNAs may be designed to target multiple target sequences in the bacterial cell(s) of interest and may be introduced into the bacterial cell(s) of interest in the form of a CRISPR array in the format direct repeat-spacer-direct repeat-spacer, etc., repeating for the number of desired spacers. In these CRISPR arrays, the direct repeat sequences represent the portion of the gRNA that is recognized by Cms1. The direct repeat is processed by Cms1 enzymes to generate mature crRNAs that associate with the Cms1 protein to form the ribonucleoprotein complex that hybridizes with the targeted sequences in the bacterial cell(s) of interest. Direct repeat sequences for use with Cms1 enzymes may take the form, for example, of one or more of the sequences set forth in SEQ ID NOs:35-40. In some embodiments, multiple guide RNAs may be designed to target multiple target sequences in the bacterial cell(s) of interest and may be introduced into the bacterial cell(s) of interest in the form of a CRISPR array in which the mature gRNAs are processed by ribozymes or by tRNA processing pathways (WO 2019/138052; Port and Bullock (2016) BioRxiv dx.doi.org/10.1101/046417).

The DNA molecule encoding the Cms1 enzyme and/or the guide RNA(s) can be linear or circular. In some embodiments, the DNA sequence encoding the Cms1 enzyme and/or the guide RNA(s) can be part of a vector. Suitable vectors include plasmid vectors (for example conjugative plasmid vectors), phagemids, cosmids, artificial/mini-chromosomes, transposons, and viral vectors. In an exemplary embodiment, the DNA encoding the Cms1 enzyme and/or the guide RNA(s) is present in a plasmid vector. Non-limiting examples of suitable plasmid vectors include pUC, pBR322, pET, pBluescript, pCAMBIA, and variants thereof. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. In another exemplary embodiment, the DNA encoding the Cms1 enzyme and/or the guide RNA(s) can be part of a phagemid.

In embodiments in which both the Cms1 polypeptide and the guide RNA(s) are introduced into the genome host as DNA molecules, each can be part of a separate molecule (e.g., one vector containing Cms1 polypeptide or fusion protein coding sequence and a second vector containing guide RNA coding sequence(s)) or both can be part of the same molecule (e.g., one vector containing coding (and regulatory) sequence for both the Cms1 polypeptide and the guide RNA(s)).

A Cms1 polypeptide in conjunction with a guide RNA is directed to a target site (i.e., a targeted DNA sequence or target sequence) in a bacterial cell, wherein the Cms1 polypeptide hybridizes with the targeted DNA sequence (the “initial hybridization event”) and produces a double-stranded break (i.e., cleavage) in the targeted DNA sequence. The cleavage site can be located anywhere within the target sequence. Without being limited by theory, this initial hybridization event triggers a conformational change in the Cms1 polypeptide that allows the Cms1 polypeptide to degrade RNA and/or dsDNA in a non-sequence-specific manner. The target site has no sequence limitation except that the sequence is immediately preceded (upstream) by a consensus sequence. This consensus sequence is also known as a protospacer adjacent motif (PAM). Examples of PAM sequences include, but are not limited to, TTTN, NTTN, TTTV, and NTTV (wherein N is defined as any nucleotide and V is defined as A, G, or C). It is well-known in the art that a suitable PAM sequence must be located at the correct location relative to the targeted DNA sequence to allow the Cms1 nuclease to produce the desired double-stranded break. For all Cms1 nucleases characterized to date, the PAM sequence is located immediately 5′ of the targeted DNA sequence. Thus, the targeted sequence is immediately downstreatm (3′) of the PAM sequence. The PAM site requirements for a given Cms1 nuclease cannot at present be predicted computationally, and instead must be determined experimentally using methods available in the art (Zetsche et al. (2015) Cell 163:759-771; Marshall et al. (2018) Mol Cell 69:146-157). It is well-known in the art that PAM sequence specificity for a given nuclease enzyme is affected by enzyme concentration (Karvelis et al. (2015) Genome Biol 16:253). Thus, modulating the concentrations of Cms1 protein delivered to the cell or in vitro system of interest represents a way to alter the PAM site requirements associated with that Cms1 enzyme. Modulating Cms1 protein concentration in the system of interest may be achieved, for instance, by altering the promoter used to express the Cms1-encoding gene, by altering the concentration of ribonucleoprotein delivered to the cell or in vitro system, or by adding or removing introns that may play a role in modulating gene expression levels. As detailed herein, the first region of the guide RNA is complementary to the protospacer of the target sequence. Typically, the first region of the guide RNA is about 19 to 25 nucleotides in length.

The target site can be in the coding region of a gene, in an intron of a gene, in a control region of a gene, in a non-coding region between genes, etc. The gene can be a protein coding gene or an RNA coding gene. The gene can be any gene of interest as described herein. Cms1 collateral activity against RNA and/or dsDNA may be activated through an in initial hybridization event with any DNA sequence(s) in the bacterial cell(s) of interest as long as a suitable PAM site is located 5′ of the target sequence(s).

In some embodiments, the Cms1 protein, or Cms1 protein-encoding polynucleotide, and guide RNA(s), or DNA encoding the guide RNA(s), are introduced into a plurality of bacterial cells with the guide RNA(s) designed to target sequences that are present only in a certain fraction of the cells. In some embodiments, this will result in the elimination or reduction of those cells that comprise the targeted sequence(s) that the guide RNA(s) are designed to hybridize with.

By “predetermined” or “targeted sequence” is intended a nucleotide (e.g., DNA or RNA) sequence in the microbe of interest that is unique to that microbe. The predetermined or targeted sequence may be genomic DNA, chromosomal DNA, and/or plasmid or other extrachromosomal DNA sequences present in the cell or cells of interest. Methods are available in the art to find unique sequences within genomes and include using a Pan-Core genome approach to find accessory genes of organisms. Additionally using a Best Bi-directional Blast analysis or using OrthoMCL etc, would identify accessory genes. Additionally, unique regions between a pair of genomes can be extracted from a pair-wise global alignment performed using any of the popular programs like Nucmer (MUMmer), Mauve, BLAST, and the like. In some embodiments, a targeted sequence of interest is a sequence that is part of an antibiotic resistance gene. Antibiotic resistance gene sequences are known in the art and include, for example and without limitation, GyrB, ParE, ParY, AAC(1), AAC(2′), AAC(3), AAC(6′), ANT(2″), ANT(3″), ANT(4′), ANT(6), ANT(9), APH(2″), APH(3″), APH(3′), APH(4), APH(6), APH(7″), APH(9), ArmA, RmtA, RmtB, RmtC, Sgm, AER, BLA1, CTX-M, KPC, SHV, TEM, BlaB, CcrA, IMP, NDM, VIM, ACT, AmpC, CMY, LAT, PDC, OXA β-lactamase, mecA, Omp36, OmpF, PIB (por), bla (blaI, blaR1) and mec (mecI, mecR1) operons, Chloramphenicol acetyltransferase (CAT), Chloramphenicol phosphotransferase, EmbB, Mupirocin-resistant isoleucyl-tRNA synthetases MupA, MupB, MprF, Cfr 23S rRNA methyltransferase, Rifampin ADP-ribosyltransferase (Arr), Rifampin glycosyltransferase, Rifampin monooxygenase, Rifampin phosphotransferase, Rifampin resistance RNA polymerase-binding proteins DnaA, RbpA, Rifampin-resistant beta-subunit of RNA polymerase (RpoB), Cfr 23S rRNA methyltransferase, Erm 23S rRNA methyltransferases (e.g., ErmA, ErmB, Erm(31)), Streptogramin resistance ATP-binding cassette (ABC) efflux pumps (e.g., Lsa, MsrA, Vga, VgaB), Streptogramin Vgb lyase, Vat acetyltransferase, Fluoroquinolone acetyltransferase, Fluoroquinolone-resistant DNA topoisomerases, Fluoroquinolone-resistant GyrA, GyrB, ParC, Quinolone resistance protein (Qnr), FomA, FomB, FosC, FosA, FosB, FosX, VanA, VanB, VanD, VanR, VanS, EreA, EreB, GimA, Mgt, Ole, MPH(2′)-I, MPH(2′)-II, MefA, MefE, Mel, sat, Sul1, Sul2, Sul3, sulfonamide-resistant FolP, TetX, TetA, TetB, TetC, Tet30, Tet31, TetM, TetO, TetQ, Tet32, Tet36, MacAB-TolC, MsbA, MsrA,VgaB, EmrD, EmrAB-TolC, NorB, GepA, MepA, AdeABC, AcrD, MexAB-OprM, mtrCDE, adeR, acrR, baeSR, mexR, phoPQ, mtrR, and other such genes known to those of skill in the art (see, e.g., McArthur et al 2013 Antimicrobial Agents and Chemotherapy 57:3348-3357). In some embodiments, a targeted sequence is present in a plasmid, for example and without limitation a sequence that is present in a pOXA-48, pKpQIL, IncFII, p202c, HI2, HI1, Il-γ, X, L/M, N, FIA, FIB, FIC, W, Y, P, A/C, T, K, B/O, pAM830, pAM831 plasmid, and other such plasmids known to those of skill in the art.

### IV. Bacterial Species of Interest

### A. Plant-Associated Bacteria

Bacterial species that grow on plants or plant material may be targeted and selectively eliminated by the compositions and methods of the present invention. Non-limiting examples of plant- or plant-material associated bacterial species of interest include Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., and Phytoplasma sp. Plant-associated bacteria may include, for example, plant pathogens, nodulating bacteria, bacteria that grow on plants and may harm humans or other animals that consume the plant material, or other bacteria. The methods of the present invention may be applied pre-harvest (i.e., during plant growth) or post-harvest, or may be applied to seeds or isolated plant cells or cell cultures, plant parts, and may be applied, for example, to leaves, flowers, seeds, roots, stems, or other plant tissues. In some embodiments, the compositions and methods of the present invention may be used to reduce the number of cells of a given bacterial strain or species, or to eliminate all or nearly all of the cells of a given bacterial strain or species. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### B. Animal-Associated Bacteria

Bacterial species that grow in or on animals or animal parts (e.g., meat, bones, teeth, organs, etc.) may be targeted and selectively eliminated by the compositions and methods of the present invention. Non-limiting examples of such animal-associated bacterial species of interest include Escherichia sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Legionella sp., Leptospira sp., Mycobacterium sp., Neisseria sp., Nocardia sp., Treponema sp., Vibrio sp., and Yersinia sp. Animal-associated bacteria may include, for example, bacteria that live in or on oral cavities, gut tissues (e.g., stomach, intestines, etc.), stool, genitalia, skin, hair, eyes, ears, nasal cavities, the bloodstream, and/or the tissues of the respiratory system, and the like. Animal-associated bacteria may also live on animal parts in dead animals, for example, in animal meat, skin, bones, organs, brain, and/or other tissues. The methods of the present invention may be applied to living animals, for example to reduce or eliminate harmful bacteria such as pathogenic bacteria that may cause health problems for the animal that harbors the bacterial cell(s) of interest. The methods of the present invention may be applied to animal parts such as, for example, meat or other products intended for consumption by humans or other animals, for example to reduce or eliminate the presence of harmful or potentially harmful bacteria such as those that may cause disease in humans or animals that consume the animal parts. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### C. Human-Associated Bacteria

Bacterial species that grow in or on humans represent a subset of those bacteria that grow in or on animals or animal parts and may be targeted and selectively eliminated by the compositions and methods of the present invention. Non-limiting examples of such human-associated bacterial species of interest include Escherichia sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., and Yersinia sp. Human-associated bacteria may include, for example, bacteria that live in or on oral cavities, gut tissues (e.g., stomach, intestines, etc.), stool, genitalia, skin, hair, eyes, ears, nasal cavities, the bloodstream, and/or the tissues of the respiratory system, and the like. The methods of the present invention may be applied therapeutically, for example to reduce or eliminate harmful bacteria such as pathogenic bacteria that may cause health problems for the human that harbors the bacterial cell(s) of interest. The compositions of the present invention may be delivered to humans through various routes of administration, for example through inhalation, ingestion, injection, or other routes of administration. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### D. Fungus-Associated Bacteria

Bacterial species that grow in close contact with fungal organisms or cells may be targeted and selectively eliminated by the compositions and methods of the present invention. For example, bacteria that interfere with fungal culture and/or fungal fermentation may be targeted for control, elimination, or reduction by the compositions and methods of the present invention. Non-limiting examples of such fungus-associated bacterial species of interest include Enterobacter sp., Pseudomonas sp., Klebsiella sp., Serratia sp., Staphylococcus sp., Escherichia sp., Clostridium sp., Enterococcus sp., and other such bacterial species. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### E. Arthropod-Associated Bacteria

Bacterial species that grow in close contact with arthropods or other insects may be targeted and selectively eliminated by the compositions and methods of the present invention. For example, some arthropods are known to harbor symbiotic bacteria that may be selectively reduced or eliminated using the compositions and methods of the present invention. Some arthropod species that may be of particular interest for use with the compositions and methods of the present invention include those that transmit disease to humans or animals (non-limiting examples include ticks and mosquitoes), those that transmit disease to plants (non-limiting examples include aphids and psyllids), and arthropods that are farmed, for example for human consumption (non-limiting examples include shrimp, crabs, and lobsters). In some embodiments, bacteria that enable disease transmission to plants, humans, or other animals by arthropods, or bacteria that are required for disease transmission to plants, humans, or other animals by arthropods, may be targeted and selectively eliminated using the compositions and methods of the present invention. In some embodiments, bacteria that contaminate cultivated aquacultural arthropods (e.g., shrimp, crabs, lobsters, and other arthropods) may be targeted and selectively eliminated using the compositions and methods of the present invention. Non-limiting examples of such arthropod-associated bacteria include Borrelia sp., Rickettsia sp., Anaplasma sp., Francisella sp., Coxiella sp., Wolbachia sp., Ehrlichia sp., Liberibacter sp., Aeromonas sp., Vibrio sp., Edwardsiella sp., Streptococcus sp., Yersinia sp., Flavobacterium sp., Tenacibaculum sp., Renibacterium sp., Piscirickettsia sp., Mycobacterium sp., Pseudomonas sp., Clostridium sp., Enterobacterium sp., Nocardia sp., Lactococcus sp., Aerococcus sp., Hepatobacter sp., Chlamydia sp., and other such bacterial species. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### F. Environmental Bacteria

Bacterial species that grow in the environment may be targeted and selectively eliminated by the compositions and methods of the present invention. Environments of particular interest may include, without limitation, wastewater, water intended for treatment to render it potable, surgical instruments and other materials in hospitals or other environments where sterility is required, and other such environments. In some embodiments, bacteria living in these and other environments may be targeted for reduction or elimination through the use of the compositions and methods of the present invention. In some embodiments, the compositions and methods of the present invention may be used to selectively target and eliminate (in whole or in part) only those cells of a given bacterial strain or species that harbor certain targeted sequences, whether those sequences are present in the bacterial chromosomal genome, in plasmids, in viruses or phages that have infected or are otherwise present in the bacteria, or in other DNA-containing components found in those bacterial cells.

### V. Enrichment of Cell Types

The compositions and methods of the present invention may be used to reduce or eliminate the presence of cells that comprise undesirable DNA sequence(s). In some embodiments, the compositions and methods of the present invention may be used to enrich for cells, cell lines, cell types, or other groupings of cells that do not comprise undesirable DNA sequence(s). Enrichment of certain cell types may be desirable, for example, following genome editing experiments or other experiments designed to modify certain known regions of a genome or other DNA molecule. In such embodiments, the genome editing experiment may be performed to produce a desired genomic modification, resulting in a pool of cells in which a portion of the cells remain wild-type while a portion of the cells comprises the desired DNA sequence modification(s). The compositions and methods of the present invention may be used to target, through the appropriate design of guide RNA(s) or other guide polynucleotides designed to hybridize with wild-type, but not with modified sequences. Introduction of a Cms1 polypeptide, or encoding polynucleotide, along with one or more appropriately designed guide RNA(s) or encoding DNA molecules, into the pool of cells (for example through the use of engineered phages or phagemids, or through the use of conjugative plasmids), results in an initial hybridization event in cells that retain the undesirable wild-type sequence(s). This initial hybridization event triggers secondary, collateral activity of the Cms1 enzyme targeted against dsDNA and/or RNA, resulting in cell death among those cells that comprise the undesirable wild-type sequence(s). The result of the targeted elimination of wild-type cells is the enrichment of cells in the cell pool that comprise the desired DNA sequence(s). Such experiments may be used, for example, to increase the likelihood of identifying and recovering cells that comprise a desirable allele or other genetic sequence, particularly in cases when such a desirable allele is relatively rare among the cells in the cell pool prior to introduction of the Cms1 polypeptide and guide RNA(s) or guide polynucleotides.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

Although the foregoing invention has been described in some detail by way of illustration and example for purposes of clarity of understanding, it will be obvious that certain changes and modifications may be practiced within the scope of the appended claims.

Embodiments of the invention include:
1. A composition comprising:

(i) a Cms1 polypeptide, or a polynucleotide encoding a Cms1 polypeptide, and

(ii) a guide polynucleotide, or a polynucleotide encoding a guide polynucleotide,

wherein said guide polynucleotide is designed to interact, and capable of interacting, with a Cms1 polypeptide and to hybridize with a targeted sequence in one or more bacterial cells of interest, wherein said targeted sequence is located immediately downstream of a PAM sequence that is recognized by said Cms1 polypeptide.
2. The composition of embodiment 1, wherein said Cms1 polypeptide shares at least 80% identity with a sequence selected from the group consisting of SEQ ID NOs:41-160 and 340-341, or is encoded by a polynucleotide that shares at least 80% identity with a sequence selected from the group consisting of SEQ ID NOs:161-317 and 342-343 and wherein said guide polynucleotide is designed to interact with a Cms1 polypeptide and to hybridize with a targeted sequence in one or more bacterial cells, wherein said targeted sequence is located immediately downstream of a PAM sequence that is recognized by said Cms1 polypeptide.
3. The composition of embodiment 1, wherein said Cms1 polypeptide comprises a sequence selected from the group consisting of SEQ ID NOs:41-160 and 340-341, or is encoded by a polynucleotide that comprises a sequence selected from the group consisting of SEQ ID NOs: 161-317 and 342-343.
4. The composition of embodiment 1, wherein said one or more bacterial cells is a pathogenic bacterial species.
5. The composition of embodiment 4, wherein said one or more bacterial cells is a pathogenic bacterial species associated with plants.
6. The composition of embodiment 4, wherein said one or more bacterial cells is a pathogenic bacterial species associated with mammals.
7. The composition of embodiment 6, wherein said one or more bacterial cells is a pathogenic bacterial species associated with humans.
8. The composition of embodiment 1, wherein said one or more bacterial cells is a cell of a bacterial species selected from the group consisting of Xanthomonas sp., Escherichia sp., Pseudomonas sp., Erwinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.
9. The composition of embodiment 1 wherein said guide polynucleotide is a guide RNA.
10. A method of killing one or more bacterial cells comprising introducing the composition of embodiment 1 into said one or more bacterial cells. The method of killing wherein said killing comprises a 5%, 10%, 15%, 20%, 25%, 30%, 50%, 75%, 80%, 85%, 90%, 95%, 96%, 97%, 98%, 99%, 100% or 5-20%, 25-50%, 50-60%, 60-75%, 50-80%, 80-90%, 80-95%, 80-99%, 90-95%, 90-99%, or more decrease in the viable bacterial population.
11. The method of embodiment 10, wherein said introducing comprises contacting said one or more bacterial cells with a phage or a phagemid engineered to comprise:

(i) a polynucleotide encoding a Cms1 polypeptide, and

(ii) a polynucleotide encoding a guide polynucleotide,

wherein said polynucleotide encoding a Cms1 polypeptide shares at least 80% identity with a sequence selected from the group consisting of SEQ ID NOs: 161-317 and 342-343, or encodes a polypeptide that shares at least 80% identity with a sequence selected from the group consisting of SEQ ID NOs:41-160 and 340-341, and wherein said guide polynucleotide is designed to interact with a Cms1 polypeptide and to hybridize with a targeted sequence in said one or more bacterial cells, wherein said targeted sequence is located immediately downstream of a PAM sequence that is recognized by said Cms1 polypeptide.
12. The method of embodiment 10 wherein said introducing comprises contacting said one or more bacterial cells with a phage or a phagemid engineered to comprise:

(i) a polynucleotide encoding a Cms1 polypeptide, and

(ii) a polynucleotide encoding a guide polynucleotide,

wherein said polynucleotide encoding a Cms1 polypeptide comprises a sequence selected from the group consisting of SEQ ID NOs: 161-317 and 342-343, or encodes a polypeptide that comprises a sequence selected from the group consisting of SEQ ID NOs:41-160 and 340-341, and wherein said guide polynucleotide is designed to hybridize with a Cms1 polypeptide and to hybridize with a targeted sequence in said one or more bacterial cells, wherein said targeted sequence is located immediately downstream of a PAM sequence that is recognized by said Cms1 polypeptide.
13. The composition of embodiment 1 wherein said composition comprises an engineered phage or phagemid.
14. The composition of embodiment 1 wherein said polynucleotide encoding a Cms1 polypeptide and said polynucleotide encoding a guide polynucleotide are part of the same polynucleotide.
15. The composition of embodiment 13 or embodiment 14 wherein said engineered phage or phagemid is derived from a phage selected from the group consisting of M13, lambda, p22, T7, Mu, T4 phage, PBSX, PlPuna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.
16. The method of embodiment 10, wherein said one or more bacterial cells is a pathogenic bacterial species.
17. The method of embodiment 16, wherein said one or more bacterial cells is a pathogenic bacterial species associated with plants.
18. The method of embodiment 16, wherein said one or more bacterial cells is a pathogenic bacterial species associated with mammals.
19. The method of embodiment 18, wherein said one or more bacterial cells is a pathogenic bacterial species associated with humans.
20. The method of embodiment 10 wherein said one or more bacterial cells is a cell of a bacterial species selected from the group consisting of Xanthomonas sp., Escherichia sp., Pseudomonas sp., Envinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.
21. The method of embodiment 11 wherein said phage or a phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.
22. The method of embodiment 11 wherein said guide polynucleotide is a guide RNA.
23. The composition of embodiment 1 wherein said PAM sequence that is recognized by said Cms1 polypeptide is selected from the group consisting of NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.
24. The method of embodiment 11 wherein said PAM sequence that is recognized by said Cms1 polypeptide is selected from the group consisting of NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.
25. The composition of embodiment 1 wherein said polynucleotide encoding a Cms1 polypeptide and said polynucleotide encoding a guide polynucleotide are part of a vector.
26. The composition of embodiment 25 wherein said vector is selected from the group consisting of phages, phagemids, and conjugative plasmids.
27. The composition of embodiment 1 wherein said Cms1 polypeptide comprises one or more amino acid motifs selected from the group consisting of SEQ ID NOs:1-34.

The following examples are offered by way of illustration and not by way of limitation.

### EXPERIMENTAL

### Example 1—Design of E. coli Transformation Vectors

A PAM plasmid library was generated by PCR-amplifying the targeted plasmid backbone pOD7 (SEQ ID NO:329) using primers Odpr0023_Cms1_PAM_F and Odpr0024_Cms1_R (Table 1). The forward primer was synthetized to contain 5-nucleotide-long NNNNN adapters at the 5′ end comprising 1,024 possible nucleotide combinations. The linear PCR products obtained with these primers were ligated to create a plasmid library. The ligation reactions were cleaned by ethanol precipitation and electroporated into E. coli TOP10 cells. Electroporation efficiency was verified by plating. The number of transformed cells exceeded the number of possible PAM combinations by approximately 2,500-fold. The recovered transformants were grown overnight with antibiotic selection (kanamycin) in 100 mL LB medium and harvested by centrifugation (4,000 g for 30 minutes). The targeted PAM plasmid library was purified from the pellet using MIDI Kit (Zymo Research, D4201) and cleaned by ethanol precipitation. The presence of every nucleotide in each PAM position was roughly verified via Sanger sequencing.

TABLE 1
Primers used for plasmid generation

| Primer Name | Sequence |
|---|---|
| Odpr0023_Cms1_PAM_F (SEQ ID NO: 330) | NNNNNGGTGAGTTCTTCTTGTTGTTCCT |
| Odpr0024_Cms1_R (SEQ ID NO: 331) | TGGAGCAACACCTGAAGGAAG |
| ODpr0055_PAM_Illumina_F (SEQ ID NO: 332) | TCGTCGGCAGCGTCAGATGTGTATAAGAGACAGatgaccactgtggcatcg |
| ODpr0056_PAM_Illumina_R (SEQ ID NO: 333) | GTCTCGTGGGCTCGGAGATGTGTATAAGAGACAGaagaatcgccaaacaac |
| | aacaa |
| ODpr0058_i5_501_F (SEQ ID NO: 334) | AATGATACGGCGACCACCGAGATCTACACtagatcgcTCGTCGGCAGCGTC |
| ODpr0059_i5_502_F (SEQ ID NO: 335) | AATGATACGGCGACCACCGAGATCTACACctctctatTCGTCGGCAGCGTC |
| ODpr0060_i7_707_R (SEQ ID NO: 336) | CAAGCAGAAGACGGCATACGAGATgtagagagGTCTCGTGGGCTCGG |
| ODpr0061_i7_708_R (SEQ ID NO: 337) | CAAGCAGAAGACGGCATACGAGATcctctctgGTCTCGTGGGCTCGG |

### Example 2—Plasmid Clearance Assays for PAM Requirement Determination

For the PAM assay, the targeted PAM plasmid library was electroporated into E. coli BL21-AI with the targeting plasmid pOD4 (SEQ ID NO:338), comprising a gene encoding the SulfCms1 nuclease as well as a crRNA designed to hybridize with the target in the PAM plasmid library. Before these cells were made electrocompetent, expression of the nuclease and the crRNA from the separate T7 promoters was induced using arabinose and IPTG. As a control reaction, a plasmid containing the SulfCms1 nuclease but no repeat-spacer sequence (pOD49 (SEQ ID NO:339)) was used. Electroporation efficiency was verified by plating. The number of transformed cells exceeded the number of possible PAM combinations by approximately 1,700 and 11,340-fold for the targeting treatment compared to the control, respectively. Plasmid DNA was purified from the overnight cultures. The plasmid locus containing the PAMs was PCR amplified using primers ODpr0055_PAM_Illumina_F and ODpr0056_PAM_Illumina_R. The PCR products were cleaned up with AMPure beads (Beckman Coulter, A63882). The cleaned PCR products were indexed for primers having the sequences described by SEQ ID NOs:334-337 (Table 1) and cleaned once more using the AMPure beads. The final DNA amount in the resulting PCR reactions was quantified using DeNovix dsDNA Broad Range Kit (DeNovix), pooled, and sent for sequencing on the Illumina MiSeq paired-end 300 platform. The sequence data was analyzed as described previously (Leenay et al (2016) Mol Cell 62:137-147). Two biological replicates per treatment were sequenced. The sequencing data showed that SulfCms1 efficiently recognized a variety of PAM sites. These PAM sites are summarized in Table 2.

TABLE 2
PAM sites recognized by SulfCms1

| | | |
|---|---|---|
| NACTV | KGCTC | RTCSV |
| NATVR | NGTRR | YTCGA |
| BATCC | NGTCV | VTCTN |
| YATGC | TGTGC | TTCTR |
| NATTN | NGTTN | NTGTV |
| NCCTR | ATARG | ATTAT |
| NCTMR | RTACR | DTTCN |
| VCTCC | NTATV | CTTCK |
| NCTKV | HTCAR | NTTRV |
| NGCTR | ATCAC | ATTGT |
| | | NTTTN |

The PAM sites listed in Table 2 that are recognized by SulfCms1 represent 288 of the 1,024 PAM sequences in the five base pair PAM library.

To validate the results of the PAM library screen, individual PAM sequences were tested in the same co-transformation screen to test whether the PAM sites could be efficiently recognized by SulfCms1. These individual assays confirmed the results of the library screen. In individual plasmid clearance assays, plasmids with CTTTC, CGCTG, CCTTC, CCTCC, CAATC, and CTGAA PAM sites were efficiently cleared, while plasmids with CAGCG and CTGGT PAM sites were not efficiently cleared.

In addition to SulfCms1, additional Cms1 enzymes were tested in these E. coli plasmid clearance assays. Table 3 summarizes the results of these plasmid clearance assays.

TABLE 3
Plasmid clearance assay results

| | SEQ ID | log |
|---|---|---|
| nuclease | NO | clearance |
| Pb2Cpf1 | 324 | 3.45 |
| Sulf | 42 | 3.27 |
| Sulf | 42 | 3.87 |
| Sulf | 42 | 2.92 |
| Sulf | 42 | 2.78 |
| Sulf | 42 | 3.12 |
| Unk34 | 82 | 3.94 |
| Unk7 | 57 | 3.24 |
| Unk7 | 57 | 2.92 |
| Unk35 | 83 | 1.87 |
| Unk35 | 83 | 1.72 |
| Unk37 | 85 | 1.57 |
| Unk33 | 81 | 1.45 |
| Unkll | 61 | 1.24 |
| Unk25 | 73 | 0.79 |
| Unkl9 | 67 | 0.65 |
| Unkl5 | 63 | 0.60 |
| Unk24 | 72 | 0.34 |
| Unk26 | 74 | 0.26 |
| Unk9 | 59 | 0.24 |
| Unk3 | 53 | 0.19 |
| Unk21 | 69 | 0.15 |
| Unk27 | 75 | 0.11 |
| Unk4 | 54 | 0.10 |
| Unk6 | 56 | 0.07 |
| Unk2 | 52 | 0.06 |
| Unkl7 | 65 | 0.06 |
| Unk41 | 89 | −0.08 |
| Unk5 | 55 | −0.10 |
| Unk40 | 88 | −0.13 |
| Unkl4 | 62 | −0.15 |
| Unkl6 | 64 | −0.22 |

The data in Table 3 show that many of the Cms1 nucleases tested in the E. coli plasmid clearance assay resulted in substantial plasmid clearance, leading to cell death in these assays.

### Example 3—Positive Selection Assays in E. coli

SulfCms1 was used in a positive selection assay in E. coli. In these assays, the guide RNA associated with SulfCms1 was designed to hybridize with a sequence in the ileS gene present in the 133908 plasmid (SEQ ID NO:318), which also comprised an arabinose/IPTG-inducible ccdB toxin gene. The ileS gene present in the 133908 plasmid comprised four base changes relative to the ileS gene present in the E. coli chromosome. Disruption of the 133908 plasmid was expected to result in cell survival in these assays by preventing the expression of the ccdB gene following induction by arabinose and IPTG.

C173 E. coli cells comprising the 133908 plasmid were grown overnight at 37° C. in liquid LB medium comprising 50 μg/mL kanamycin. Following overnight growth, three replicate 5 mL cultures of LB medium comprising 50 μg/mL kanamycin were each inoculated with 100 μL of the overnight culture. These 5 mL cultures were grown for four hours at 37° C. to an OD₆₀₀ₙₘ of approximately 0.6. These cell cultures were then centrifuged at 5,000 rpm for five minutes at room temperature to pellet the cells, after which the supernatant was discarded. The cell pellets were then combined in 1 mL of molecular biology-grade water (dH₂O) and transferred to a 1.7 mL microcentrifuge tube. The cells were pelleted and washed twice, with pelleting accomplished by spinning at 9,000 rpm for one minute followed by a wash in 1 mL dH₂O. The washed cell pellets were then resuspended in 262.5 μL dH₂O, and 50 μL aliquots were prepared for transformation. The cells were transformed via electroporation with 100 ng of plasmid 133956 (SEQ ID NO:319) or 134136 (SEQ ID NO:320), each comprising a chloramphenicol resistance gene and designed to express SulfCms1, along with a guide RNA designed to hybridize with the iles target (134136) or a negative control guide RNA designed to target a sequence not present in these experiments (133956). Duplicate transformations were performed with each plasmid. The cells were allowed to recover in 5004, of SOC medium by shaking for one hour at 37° C. Following recovery, 1004, of log-dilutions from 10° to 10′ were plated on LB agar plates comprising chloramphenicol and allowed to grow overnight at 37° C. CFU (colony forming units) were counted the following day. Table 4 shows the number of colonies obtained from each dilution in this experiment, with each replicate on a separate row:

TABLE 4
CFU observed following ileS-targeting positive selection results

| | Nuclease/ | | | | |
|---|---|---|---|---|---|
| Target | Guide | 10⁰ | 10⁻¹ | 10⁻² | 10⁻³ |
| Plasmid | Plasmid | Dilution | Dilution | Dilution | Dilution |
| 133908 | 133956 | Lawn | Too many | 164 | 15 |
| | | | to count | | |
| | | Lawn | Too many | 254 | 23 |
| | | | to count | | |
| | 134136 | 2 | 1 | 0 | 0 |
| | | 11 | 0 | 0 | 0 |

As Table 4 shows, transformation with the 133956 plasmid, as expected, had no apparent effect on the cells, with many colonies surviving (ccdB gene expression was not activated in this experiment because no arabinose or IPTG was included in the cell growth media). Unexpectedly, however, transformation with the 134136 plasmid led to cell death, with few cells surviving. Because the results from the experiment summarized in Table 4 were unexpected, a follow-up experiment was performed using the same protocol with minor changes: the target plasmid used in these experiments was 133870 (SEQ ID NO:321), which comprised the same ileS target found in the 133908 plasmid, but did not include a ccdB toxin gene to avoid any complications caused by the possibility of leaky ccdB expression even in the absence of arabinose or IPTG, and an additional nuclease and guide plasmid was used in this experiment. Nuclease/guide plasmid 133938 (SEQ ID NO:322) was identical to 134136 except that the encoded nuclease was Pb2Cpf1 (SEQ ID NO:324, encoded by SEQ ID NO:323) rather than SulfCms1. Table 5 summarizes the results of these experiments.

TABLE 5
CFU observed following ileS-targeting positive selection.

| | Nuclease/ | | | | |
|---|---|---|---|---|---|
| Target | Guide | 10⁰ | 10⁻¹ | 10⁻² | 10⁻³ |
| Plasmid | Plasmid | Dilution | Dilution | Dilution | Dilution |
| 133870 | 133956 | Lawn | Lawn | Too Many | 283 |
| | | | | to Count | |
| | | Lawn | Lawn | Too Many | 242 |
| | | | | to Count | |
| | 134136 | 173 | 23 | 2 | 0 |
| | | 179 | 16 | 0 | 0 |
| | 133938 | Lawn | Lawn | Too Many | 162 |
| | | | | to Count | |
| | | Lawn | Lawn | Too Many | 168 |
| | | | | to Count | |

Consistent with the results shown in Table 4, the results in Table 5 showed that E. coli transformation with 134136, expressing SulfCms1 with a plasmid-targeting guide RNA, resulted in cell death while transformation with 133956 comprising a non-targeting guide RNA did not result in cell death. Transformation with the 133938 plasmid, expressing Pb2Cpf1, did not result in cell death.

These assays were again repeated with target plasmids 133137 (SEQ ID NO:325) and 133167 (SEQ ID NO:326). Plasmid 133137 comprises a target sequence downstream from a TTTC PAM site that is efficiently recognized by SulfCms1, while 133167 comprises the same target sequence downstream from a TGGT PAM site that is not efficiently recognized by SulfCms1. Both of these target plasmids comprise a target that was not derived from an E. coli chromosomal target that would be a likely off-target based on off-target effects observed in other CRISPR nuclease systems. Table 6 summarizes the results of these experiments.

TABLE 6
CFU observed with targeted vs. Non-Targeted PAM sequence

| | Nuclease/ | | | | |
|---|---|---|---|---|---|
| Target | Guide | 10⁰ | 10⁻¹ | 10⁻² | 10⁻³ |
| Plasmid | Plasmid | Dilution | Dilution | Dilution | Dilution |
| 133167 | 133956 | Too Many | 80 | 4 | 0 |
| | | to Count | | | |
| | | Too Many | 108 | 8 | 0 |
| | | to Count | | | |
| | 134136 | Too Many | 90 | 4 | 0 |
| | | to Count | | | |
| | | Too Many | 129 | 7 | 0 |
| | | to Count | | | |
| 133137 | 133956 | 2 | 0 | 0 | 0 |
| | | 9 | 1 | 0 | 0 |
| | 134136 | Too Many | 110 | 12 | 0 |
| | | to Count | | | |
| | | Too Many | 60 | 4 | 0 |
| | | to Count | | | |

The results summarized in Table 6 show that cell death results when SulfCms1 was expressed with a guide RNA designed to hybridize with a target sequence present in a plasmid, but only when the target sequence was located downstream from a PAM sequence that is recognized by SulfCms1. Because the target sequences present in these plasmids do not have corresponding E. coli chromosomal targets, it was concluded that the observed cell death was unlikely a result of “off-target effects” known to result when a CRISPR nuclease complex hybridizes with a target sequence that imperfectly base pairs with the associated guide RNA. Instead, it was hypothesized that a different mechanism was responsible for the cell death.

These assays were repeated with target plasmid 133870 and several nuclease/guide plasmid variations. In addition to plasmids 133956 and 134136, described above, plasmids 134396 (SEQ ID NO:328) and 134395 (SEQ ID NO:327) were also used. The 134396 plasmid comprises a disrupted SulfCms1 coding sequence encoding a non-functional SulfCms1 protein comprising only 48 amino acids. The 134395 plasmid comprises a RuvCI mutant SulfCms1 coding sequence comprising a D to A mutation in the RuvCI catalytic residue. Table 7 summarizes the results of these experiments.

TABLE 7
CFU observed with functional vs. Non-Functional SulfCmsl

| | Nuclease/ | | | | |
|---|---|---|---|---|---|
| Target | Guide | 10⁰ | 10⁻¹ | 10⁻² | 10⁻³ |
| Plasmid | Plasmid | Dilution | Dilution | Dilution | Dilution |
| 133870 | 133956 | Lawn | Too Many | 139 | 20 |
| | | | to Count | | |
| | | Lawn | Too Many | 222 | 29 |
| | | | to Count | | |
| | | Lawn | Too Many | 144 | 17 |
| | | | to Count | | |
| | | Lawn | 264 | 66 | 5 |
| | 134396 | Lawn | Too Many | 61 | 8 |
| | | | to Count | | |
| | | Lawn | Too Many | 221 | 29 |
| | | | to Count | | |
| | 134136 | 13 | 0 | 0 | 0 |
| | | 1 | 0 | 0 | 0 |
| | | 3 | 0 | 0 | 0 |
| | | 1 | 0 | 0 | 0 |
| | 134395 | Lawn | Too Many | 140 | 21 |
| | | | to Count | | |
| | | Lawn | Too Many | 231 | 30 |
| | | | to Count | | |

The results in Table 7 show that cell death resulted when a functional SulfCms1 protein was present with a guide RNA designed to hybridize with a target sequence, but not when a non-functional protein (134396 plasmid) or a RuvCI mutant SulfCms1 protein (134395 plasmid) were present.

The results summarized in Tables 4 through 7 collectively show that SulfCms1, but not Pb2Cpf1, effectively kills E. coli cells in a sequence specific manner that is not consistent with “off-target effects” that are known to occur with CRISPR nucleases. Importantly, cell death resulted even when non-essential genes on plasmids were targeted by SulfCms1, and resulted only when a functional SulfCms1 protein comprising a functional RuvC domain was present and when a PAM sequence recognized by SulfCms1 was present upstream of the targeted sequence.

### Example 4—Phagemid-Encoded Cms1 Cell Killing

Genes encoding SulfCms1 (SEQ ID NO:42, encoded by SEQ ID NO:348) and LbCpf1 (SEQ ID NO:349, encoded by SEQ ID NO:350) were cloned into a modified pBAD24 plasmid vector backbone; the ampicillin resistance gene in pBAD24 for these nuclease plasmids was replaced by a chloramphenicol resistance cassette. The PJ23105 promoter (SEQ ID NO:351) was used to drive expression of the nuclease in these nuclease plasmids. The full sequences of the SulfCms1 (pOD786) and LbCpf1 (pOD818) plasmids are SEQ ID NOs: 352 and 353, respectively. The pOD789 (SEQ ID NO:346) and pOD821 (SEQ ID NO:347) target plasmids were generated in a pBAD24 plasmid backbone comprising an ampicillin resistance cassette. The pOD789 plasmid was used as a non-targeting control as the target was located downstream from a TAGAT PAM site that is not accessible by SuCms1 or LbCpf1, while the pOD821 comprised the same target sequence downstream from a ACTTA PAM site that is accessible by these nucleases. Both the pOD789 and pOD821 plasmids also comprised an identical guide RNA (SEQ ID NO:344), expressed from a synthetic promoter (SEQ ID NO:345) and designed to hybridize with the target sequence.

For preparation of phage particles comprising the nuclease and guide RNA cassettes, E. coli cells of the ER2738 strain were transformed with the relevant nuclease plasmids described above (i.e., pOD786 or pOD818) and grown in LB medium supplemented with chloramphenicol. M13K07 (New England Biolabs) helper phage was added and following a one-hour incubation, kanamycin was added to the medium. Cells were harvested to collect the packaged phage comprising the appropriate nuclease cassette and the phage were then appropriately diluted.

To test the ability of SuCms1 to mediate cell killing when expressed from a phage, E. coli cells of the EMG2 strain were transformed with either pOD789 (non-target plasmid) or pOD821 (target plasmid) and grown in LB medium supplemented with ampicillin. Cells were collected at an OD600 of approximately 0.8 and resuspended to 10⁸ CFU/mL. Appropriately diluted phage was added to this cell resuspension and incubated for 30 minutes at room temperature before the addition of the appropriate antibiotics. Chloramphenicol was added to select only for the nuclease, or a combination of chloramphenicol and ampicillin was added to select for both the nuclease and target plasmid.

Table 8 shows the number of CFUs following phage infection with phage derived from pOD786 or pOD818 in cells harboring either the pOD789 or pOD821 plasmid, then plated on LB medium comprising either chloramphenicol or a combination of chloramphenicol and ampicillin.

TABLE 8
CFU observed following phage infection

| | | | Chloramphenicol + | |
|---|---|---|---|---|
| | Chloramphenicol | | Ampicillin | |
| Phagemid | pOD789 | pOD821 | pOD789 | pOD821 |
| pOD786 | 101000 ± 11690 | 6175 ± 634 | 94500 ± 5745 | 4850 ± 436 |
| pOD818 | 109250 ± 20172 | 124750 ± 9069 | 128750 ± 7676 | 57.5 ± 38 |

The data in table 8 show that pOD786, comprising a gene encoding SulfCms1, results in robust cell death when the cells are plated on either chloramphenicol or a combination of chloramphenicol and ampicillin, while pOD818, comprising a gene encoding LbCpf1, results in robust cell death only when plated on a combination of chloramphenicol and ampicillin. Thus, similar to the results of the plasmid assays described above, SulfCms1 can mediate robust cell death following targeting of a plasmid even when that plasmid is not selected for via antibiotics, while LbCpf1 mediates cell death following plasmid targeting only when the targeted plasmid comprises an antibiotic resistance gene that is selected for.

### Example 5—Sequence Analyses of Cms1 Nucleases

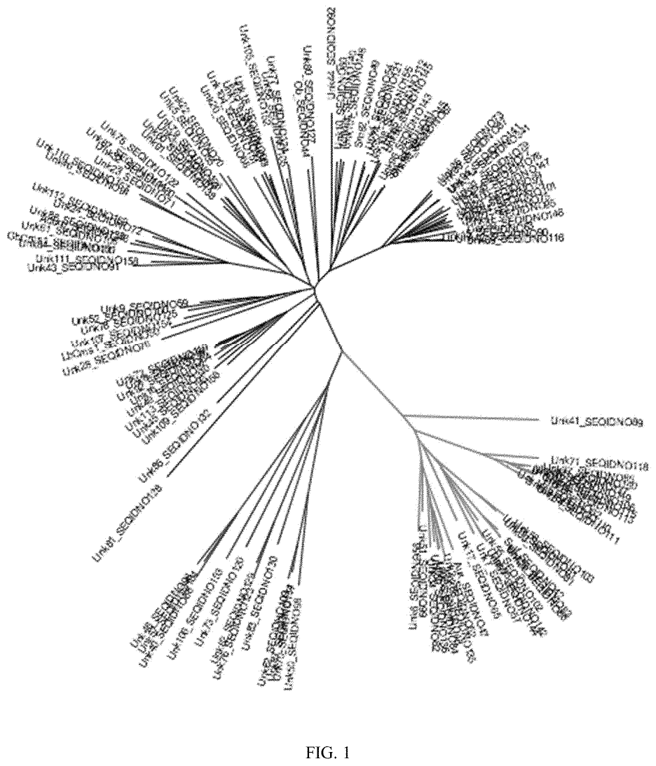

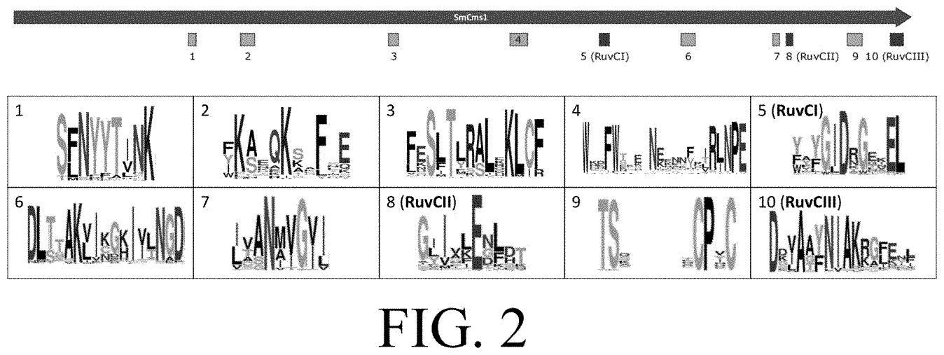

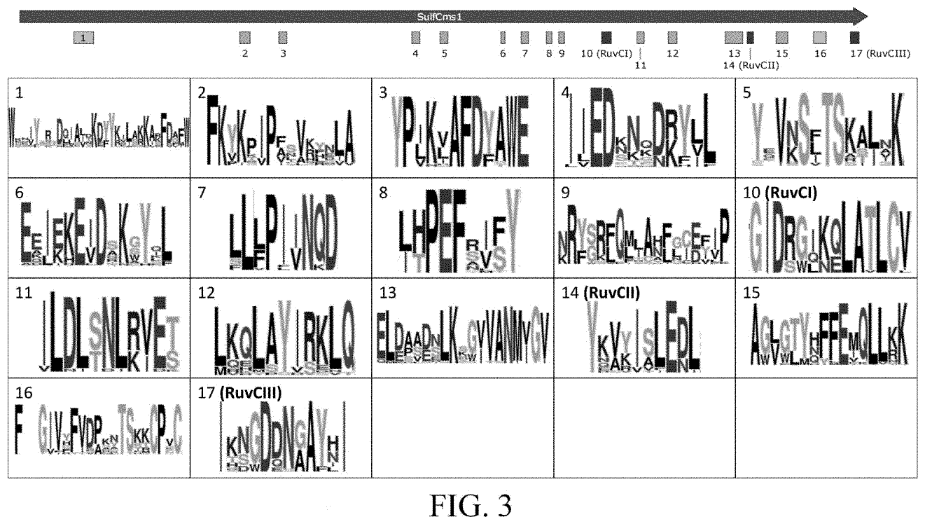

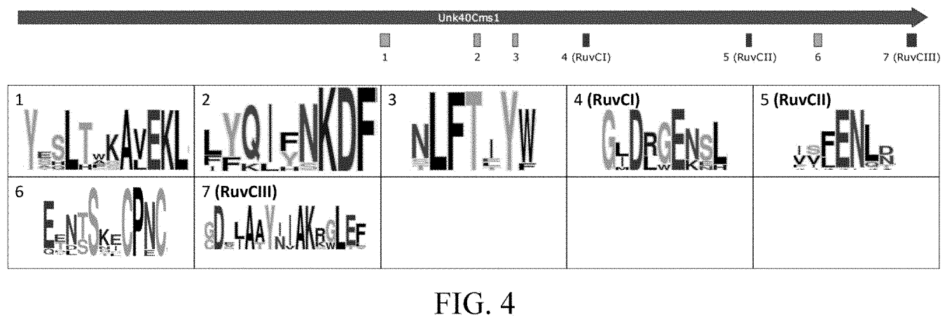

Cms1 nuclease amino acid sequence alignments were examined to identify motifs within the protein sequences that are well-conserved among these nucleases. It was observed that Cms1 nucleases were found in three fairly well-separated clades on the phylogenetic tree shown in FIG. 1. One of these clades includes SmCms1 (SEQ ID NO:41), another includes SulfCms1 (SEQ ID NO:42), and another includes Unk40Cms1 (SEQ ID NO:88). Members of each of these clades were therefore aligned separately to identify partially and/or completely conserved amino acid motifs among these nucleases. SmCms1-like nucleases include those proteins listed in the group consisting of SEQ ID NOs:41, 43-46, 49-51, 53-55, 58-60, 63, 64, 66-80, 87, 90-95, 97, 100, 101, 105, 107, 108, 112, 114, 116, 119, 121, 122, 124-128, 131-133, 138-141, 143, 145, 146-148, 151-160, and 340-341. SulfCms1-like nucleases include those proteins listed in the group consisting of SEQ ID NOs:42, 47, 48, 52, 56, 57, 61, 62, 65, 81-86, 89, 99, 102, 103, 106, 110, 111, 113, 115, 118, 135-137, 142, 144, 149, and 150. Unk40Cms1-like nucleases include those proteins listed in the group consisting of SEQ ID NOs:88, 96, 98, 104, 109, 117, 120, 123, 129, 130, and 134. The amino acid motifs shown in SEQ ID NOs:1-10 were identified from the alignment of SmCms1-like nucleases; the amino acid motifs shown in SEQ ID NOs:11-27 were identified from the alignment of SulfCms1-like nucleases; the amino acid motifs shown in SEQ ID NOs:28-34 were identified from the alignment of Unk40Cms1-like nucleases. Weblogos were created using the sequence alignments and are depicted graphically in FIGS. 2-4 (SmCms1-like, SulfCms1-like, and Unk40Cms1-like sequence motifs, respectively; weblogo.berkeley.edu) along with schematic diagrams showing the locations of these conserved motifs on the SmCms1, SulfCms1, and Unk40Cms1 protein sequences.

We claim:

1. A composition comprising:
(i) a Cms1 polypeptide, or a polynucleotide encoding a Cms1 polypeptide, and
(ii) a guide polynucleotide, or a polynucleotide encoding a guide polynucleotide,
wherein said guide polynucleotide is designed to interact with a Cms1 polypeptide and to hybridize with a targeted sequence in one or more bacterial cells of interest, wherein said targeted sequence is located immediately downstream of a PAM sequence that is recognized by said Cms1 polypeptide.

2. The composition of claim 1, wherein said Cms1 polypeptide shares at least 80% identity with a sequence selected from the group consisting of SEQ ID NOs:41-160 and 340-341, or is encoded by a polynucleotide that shares at least 80% identity with a sequence selected from the group consisting of SEQ ID NOs:161-317 and 342-343 and wherein said guide polynucleotide is designed to interact with a Cms1 polypeptide and to hybridize with a targeted sequence in one or more bacterial cells, wherein said targeted sequence is located immediately downstream of a PAM sequence that is recognized by said Cms1 polypeptide.

3. The composition of claim 1, wherein said Cms1 polypeptide comprises a sequence selected from the group consisting of SEQ ID NOs:41-160 and 340-341, or is encoded by a polynucleotide that comprises a sequence selected from the group consisting of SEQ ID NOs: 161-317 and 342-343.

4. The composition of claim 1, wherein said one or more bacterial cells is a pathogenic bacterial species.

5. The composition of claim 4, wherein said one or more bacterial cells is a pathogenic bacterial species associated with plants.

6. The composition of claim 4, wherein said one or more bacterial cells is a pathogenic bacterial species associated with mammals.

7. The composition of claim 6, wherein said one or more bacterial cells is a pathogenic bacterial species associated with humans.

8. The composition of claim 1, wherein said one or more bacterial cells is a cell of a bacterial species selected from the group consisting of Xanthomonas sp., Escherichia sp., Pseudomonas sp., Envinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

9. The composition of claim 1 wherein said guide polynucleotide is a guide RNA.

10. A method of killing one or more bacterial cells comprising introducing the composition of claim 1 into said one or more bacterial cells.

11. The method of claim 10, wherein said introducing comprises contacting said one or more bacterial cells with a phage or a phagemid engineered to comprise:
(i) a polynucleotide encoding a Cms1 polypeptide, and
(ii) a polynucleotide encoding a guide polynucleotide,
wherein said polynucleotide encoding a Cms1 polypeptide shares at least 80% identity with a sequence selected from the group consisting of SEQ ID NOs: 161-317 and 342-343, or encodes a polypeptide that shares at least 80% identity with a sequence selected from the group consisting of SEQ ID NOs:41-160 and 340-341, and wherein said guide polynucleotide is designed to interact with a Cms1 polypeptide and to hybridize with a targeted sequence in said one or more bacterial cells, wherein said targeted sequence is located immediately downstream of a PAM sequence that is recognized by said Cms1 polypeptide.

12. The method of claim 10 wherein said introducing comprises contacting said one or more bacterial cells with a phage or a phagemid engineered to comprise:
(i) a polynucleotide encoding a Cms1 polypeptide, and
(ii) a polynucleotide encoding a guide polynucleotide,
wherein said polynucleotide encoding a Cms1 polypeptide comprises a sequence selected from the group consisting of SEQ ID NOs: 161-317 and 342-343, or encodes a polypeptide that comprises a sequence selected from the group consisting of SEQ ID NOs:41-160 and 340-341, and wherein said guide polynucleotide is designed to hybridize with a Cms1 polypeptide and to hybridize with a targeted sequence in said one or more bacterial cells, wherein said targeted sequence is located immediately downstream of a PAM sequence that is recognized by said Cms1 polypeptide.

13. The composition of claim 1 wherein said polynucleotide encoding a Cms1 polypeptide and said polynucleotide encoding a guide polynucleotide are part of a vector.

14. The composition of claim 13 wherein said vector is selected from the group consisting of phages, phagemids, and conjugative plasmids

15. The composition of claim 1 wherein said polynucleotide encoding a Cms1 polypeptide and said polynucleotide encoding a guide polynucleotide are part of the same polynucleotide.

16. The composition of claim 14 wherein said engineered phage or phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.

17. The method of claim 10, wherein said one or more bacterial cells is a pathogenic bacterial species.

18. The method of claim 17, wherein said one or more bacterial cells is a pathogenic bacterial species associated with plants.

19. The method of claim 17, wherein said one or more bacterial cells is a pathogenic bacterial species associated with mammals.

20. The method of claim 19, wherein said one or more bacterial cells is a pathogenic bacterial species associated with humans.

21. The method of claim 10 wherein said one or more bacterial cells is a cell of a bacterial species selected from the group consisting of Xanthomonas sp., Escherichia sp., Pseudomonas sp., Envinia sp., Xylella sp., Clavibacter sp., Ralstonia sp., Pectobacterium sp., Streptomyces sp., Burkholderia sp., Phytoplasma sp., Acidovorax sp., Pantoea sp., Agrobacterium sp., Spiroplasma sp., Candidatus Liberibacter sp., Dickeya sp., Serratia sp., Sphingomonas sp., Rhizobacter sp., Rhizomonas sp., Xylophilus sp., Rickettsia sp., Bacillus sp., Clostridium sp., Arthrobacter sp., Curtobacterium sp., Leifsonia sp., Rhodococcus sp., Phytoplasma sp., Enterobacter sp., Citrobacter sp., Klebsiella sp., Hafnia sp., Corynebacterium sp., Mycoplasma sp., Serratia sp., Pasteurella sp., Proteus sp., Campylobacter sp., Salmonella sp., Pseudomonas sp., Brucella sp., Staphylococcus sp., Streptococcus sp., Trueperella sp., Clostridium sp., Listeria sp., Anthrax sp., Bartonella sp., Capnocytophaga sp., Streptobacillus sp., Rickettsia sp., Anaplasma sp., Shigella sp., Borrelia sp., Actinomyces sp., Bacteroides sp., Bordetella sp., Chlamydia sp., Chlamydophila sp., Ehrlichia sp., Enterococcus sp., Francisella sp., Haemophilus sp., Helicobacter sp., Klebsiella sp., Legionella sp., Leptospira sp., Mycobacterium sp., Neisseria sp., Nocardia sp., Treponema sp., Vibrio sp., Yersinia sp., Coxiella sp., Wolbachia sp., Liberibacter sp., Aeromonas sp., Edwardsiella sp., Flavobacterium sp., Tenacibaculum sp., Renibacterium sp., Piscirickettsia sp., Enterobacterium sp., Lactococcus sp., Aerococcus sp., and Hepatobacter sp.

22. The method of claim 12 wherein said phage or a phagemid is derived from a phage selected from the group consisting of M13, lambda, p22, T7, Mu, T4 phage, PBSX, P1Puna-like, P2, 13, Bcep 1, Bcep 43, Bcep 78, T5 phage, phi, C2, L5, HK97, N15, T3 phage, P37, MS2, Q.beta., or Phi X 174, T2 phage, T12 phage, R17 phage, M13 phage, G4 phage, Enterobacteria phage P2, P4 phage, N4 phage, Pseudomonas phage .PHI.6, .PHI.29 phage and 186 phage.

23. The method of claim 10 wherein said guide polynucleotide is a guide RNA.

24. The composition of claim 1 wherein said PAM sequence that is recognized by said Cms1 polypeptide is selected from the group consisting of NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.

25. The method of claim 11 wherein said PAM sequence that is recognized by said Cms1 polypeptide is selected from the group consisting of NACTV, NATVR, BATCC, YATGC, NATTN, NCCTR, NCTMR, VCTCC, NCTKV, NGCTR, KGCTC, NGTRR, NGTCV, TGTGC, NGTTN, ATARG, RTACR, NTATV, HTCAR, ATCAC, RTCSV, YTCGA, VTCTN, TTCTR, NTGTV, ATTAT, DTTCN, CTTCK, NTTRV, ATTGT, and NTTTN.

26. The composition of claim 1 wherein said Cms1 polypeptide comprises one or more amino acid motifs selected from the group consisting of SEQ ID NOs:1-34.
